(19) United States
(12) Patent Application Publication
(10) Pub. No.: US 2004/0224857 A1
(43) Pub. Date: Nov. 11, 2004

(54) Method and composition for controlling galling

(76) Inventor: Hendrik John, Celle (DE)

Correspondence Address: CANTOR COLBURN LLP, 55 Griffin Road South, Bloomfield, CT, 06002, US

(21) Appl. No.: 10/805,400

(22) Filed: Mar. 19, 2004

Related U.S. Application Data
(60) Provisional application No. 60/455,945, filed on Mar. 19, 2003.

(51) Int. Cl. C10M 125/00; A61L 2/00; B05D 3/00

(52) U.S. Cl. 508/154; 508/175; 427/421.1; 427/458; 427/2.31

(57) ABSTRACT

A method for controlling galling including applying a composition of about 3.5 to about 36 wt %, based upon the total weight of the composition of a fluoridic or oxidic compound of a transition of alkaline earth metal; about 4 to about 96.5 wt %, based upon the total weight of the composition, of an adherent and about 0 to about 65 wt %, based upon the total weight of the composition, of a solvent contact area of a member.

An antigalling coating composition having about 3.5 to about 36 wt %, based upon the total weight of the composition, of a fluoridic or oxidic compound of a transition or alkaline earth metal; about 4 to about 96.5 wt %, based upon the total weight of the composition, of an adherent and about 0 to about 65 wt %, based upon the total weight of the composition, of a solvent.

### CROSS REFERENCE TO RELATED APPLICATIONS

This application claims the benefit of an earlier filing date from U.S. Provisional Application Ser. No. 60/455,945 filed Mar. 19, 2003, the entire disclosure of which is incorporated herein by reference.

### BACKGROUND

Devices utilizing threads or interference fit connections (or other metal to metal contact arrangements), particularly those employing high torque or stress factors, can be subject to galling. Several prior art methods of controlling galling have been attempted in the hydrocarbon exploration and recovery art, some successfully, but all are costly from a manufacturing, and/or rig time perspective. Some methods require facilities (such as for copper plating) that are not available in many parts of the world thus further complicating employment of those methods in such regions. In addition, some of the current methods, or lack of application of any method due to cost or logistics leaves many devices subject to galling, which can of course be detrimental to operations employing such devices. In view of the lack of optimal available anti-galling procedures and products, the art is in need of further options.

### SUMMARY

A method for controlling galling including applying a composition of about 3.5 to about 36 wt %, based upon the total weight of the composition of a fluoridic or oxidic compound of a transition of alkaline earth metal; about 4 to about 96.5 wt %, based upon the total weight of the composition, of an adherent and about 0 to about 65 wt %, based upon the total weight of the composition, of a solvent to a contact area of a member.

A gall resistant threaded member having a threaded member and a coating disposed at threads of the threaded member, the coating containing 90 wt %, based upon the total weight of the composition, of a fluoridic or oxidic compound of a transition or alkaline earth metal and 10 wt %, based upon the total weight of the composition, of an adherent.

An antigalling coating composition having about 3.5 to about 36 wt %, based upon the total weight of the composition, of a fluoridic or oxidic compound of a transition or alkaline earth metal; about 4 to about 96.5 wt %, based upon the total weight of the composition, of an adherent and about 0 to about 65 wt %, based upon the total weight of the composition, of a solvent.

An antigalling coating is disclosed herein having an effective amount of a fluoridic or oxidic compound of a transition of alkaline earth metal to reduce galling, an effective amount of an adherent to adhere the composition to a target surface and an effective amount of a solvent to facilitate application of the composition.

### BRIEF DESCRIPTION OF THE DRAWINGS

Referring now to the drawings wherein like elements are numbered alike in the several figures:

FIG. 1 is a schematic exploded representation of a pin thread and a box thread upon which the compositions and methods described herein are employed;

FIG. 2 is an enlarged view of a portion of FIG. 1 circumscribed by line 2-2 in FIG. 1; and

FIG. 3 is a flow diagram of a process by which the coating hereof is applied via a spray technique.

### DETAILED DESCRIPTION

In order to overcome the drawbacks inherent in anti-galling methods of the prior art, the present disclosure teaches the employment of a coating, a thread dope or both together wherein the coating and thread dope have certain chemical and physical properties. It is noted initially that all ranges disclosed herein are inclusive and combinable (e.g., ranges of up to about 25 wt % with about 5 wt % to about 20 wt % desired, would therefore include the end points and all values of the range of about 5 wt % to about 25 wt %, etc.) Both the coating and the thread dope contain an effective amount of a fluoridic or oxidic compound of a transition or alkaline earth metal to produce the desired anti-galling effect. For example, a fluoride compound such as Calcium Fluoride (CaF₂), Strontium Fluoride (SrF₂), Barium Fluoride (BaF₂), Lithium Fluoride (LiF) and Cerium Fluoride (CeF₃) may be employed as disclosed herein. These compounds and other similar compounds including those containing at least one of the foregoing materials have relatively high melting points, have brittle-elastic properties at room temperature, but assume a relatively plastic property at higher temperature. It is these properties that enable the coating and or treated dope to function well as described herein. More specifically, due to the high torque, load, as well as friction conditions of larger threaded devices, the particles of CaF₂, etc. become plastic and allow connection of a joint while achieving the desired coefficient of friction. Additionally, the particles have a high load bearing capability, which keeps the thread surfaces on the opposite connectors separated from one another. By so separating the components, galling is effectively avoided.

In addressing the coating composition, the fluoridic or oxidic component is in a ratio of about 3.5 wt % to about 36 wt %, based upon the total weight of the composition. This is solid at room temperature and pressure and is ground to a particle size of less than about 70 micrometers for use herein. This is combined with an adherent in a ratio of about 4 to about 96.5 wt %, based upon the total weight of the composition. The coating further contains a solvent in a ratio of about 0 to about 65 wt %, based upon the total weight of the composition. In one specific embodiment, the coating comprises 36 wt % calcium fluoride (CaF₂) (solid), 4 wt % of a phenolic resin as an adherent and 60 wt % of isopropyl alcohol as a solvent when in a form ready for application (spray for these ratios). It should be noted that after application, the solvent would largely dissipate leaving about 90 wt % solid and 10 wt % adherent for the hardened coating. In the particular embodiment illustrated, the target coefficient of friction is about 0.08μ (mu). This is desirable for applications utilizing a high makeup/break torque such as connecting drill pipe for a subsurface run, or other extremely high torque connections or other long sliding makeup connections. More or less CaF₂ (or other noted materials) may be employed for differing coefficients of friction as desired. It should be noted that depending upon the desired means of applying the coating and viscosity of the resin, solvent may or may not be needed. The less solvent that is used, the higher the viscosity of the composition. Therefore, if a spray-on method for application is selected, a solvent is likely to also be selected to thin the composition thus rendering a spray function more plausible and reliable. If another form of application is selected, less or no solvent might be selected. This would allow a thicker coat of the composition to be applied in a single application such as by brushing or dipping. It is to be understood that the term “thicker” is a relative term as used herein because it is necessary to apply a uniform and thin coating to the contact areas to avoid distortion of the thread profile. In the specific embodiment described, the coating is applied via spray for ease of application and handling. It will be appreciated that other methods of application may also be effective beyond those noted such as electrostatic coating, electrophoresis, etc. Electrophoresis creates a very uniform and complete coating and is therefore a desirable method by which to apply the coating disclosed herein except for the fact that infrastructure is not ubiquitously available for that method while methods such as spraying can be undertaken anywhere. Such spray or other application means may be performed most anywhere; a substantial benefit to the art. Job site applications are intended and serve to simplify what previously was an extended process.

The coating composition, both broadly and specifically as stated above, hardens due to cross-linking shortly after application and adheres to a surface to which it is applied due to the incorporation of the adherent, in the specific embodiment, the resin. Operable resins include phenolic resins, silicone (siloxane) resins, epoxy resins, epoxy novolac resins, polyimide resins, polysulfide resins, polyester, vinylester, and polyurethane. Other adherents including combinations having as a component at least one of the foregoing may be substituted without departing from the scope of the invention. Further, other adherents having similar properties may also be used. The hardening property of the coating composition is helpful in that once hardened, handling of the component requires no special care and in addition, in the hardened condition, the coating cannot be squeezed from a high contact stress area of the mating parts occasioned by a high spot thereon. In this embodiment, the shear strength of the coating is about 10 Mega Pascals (MPa) or more. Thus, the coating is effective in reducing galling even in very high contact stress regions of mating parts. The coating may be applied to any surface requiring the amelioration of galling. In addition to the foregoing, one of skill in the art may appreciate that conductive components are omitted from at least the specific example of the coating. This effectively renders the coating non-conductive which is desirable for some applications.

Turning to the thread dope portion of the disclosure, the thread dope contains the same type of fluoridic or oxidic compounds of transition and alkaline earth metals disclosed above in concentrations about 10 wt % based upon the total weight of the composition to about 50 wt % based upon the total weight of the composition. Oxides may be added in amounts of about 1 to about 10 wt % based upon the total weight of the composition to have an effect such as burnishing the contacting surfaces using the abrasive nature of the oxides. It is to be noted that care is required to avoid too much abrasive activity so that excessive material is not removed. More specifically, it is desirable to clean the contact surface but not to degrade the surface. The selected amount of oxidic material will also affect the coefficient of friction and therefore the amount selected may be used for adjustment of that property as well. Silicates may also be added in amounts of about 1 to about 15 wt % based upon the total weight of the composition to further adjust the coefficient of friction of the dope while also acting as a solid lubricant. In one embodiment, a grease such as commercially available mineral oils and thickeners (metal soaps, calcium-complexes, lithium-complexes, etc.), synthetic hydrocarbons, polyglycol, silicon, ester oils, etc. will act as a grease base for the foregoing materials and is present in an amount of about 60 wt % based upon the total weight of the composition. In one embodiment, about 30 wt % of the fluoridic or oxidic compounds of transition and alkaline earth metals are employed based upon the total weight of the composition in the grease base, one example of which uses calcium fluoride as that component. In one embodiment, the thread dope is brushed onto a surface to be mated. It will be understood however that other methods of application of the thread dope are also effective such as by spray, dipping, pouring, etc. The dope may provide lubricating properties but in one embodiment does not reduce the coefficient of friction of the mating parts (coated or not). A desired target coefficient of friction is about 0.08μ (mu). It is noted that in the grease too, the fluoridic and oxidic compounds are ground to less than about 70 μm (micrometers) in particle size. The solid material is helpful to anti-galling properties due to the ability to hold mating parts away from each other with action similar to that of the coating discussed above. Since contact is in large part prevented, so too is galling. As in the coating composition, the thread dope is, in one embodiment, non-conductive.

In further embodiments hereof addressing both the coating and the dope an additional component is added for corrosion resistance. The corrosion resistant material or compound of materials is added to the coating or dope in amounts of about 0.5 to about 10 wt % based on the total weight of the composition for the coating and about 0.5 to about 5 wt % based upon the total weight of the composition for the dope. Suitable corrosion inhibiting materials include organic compounds, inorganic compounds and metals, such as zinc containing materials or molybdenum containing materials. In one particular embodiment, the corrosion-inhibiting component is ZnSiO3 (zinc silicate).

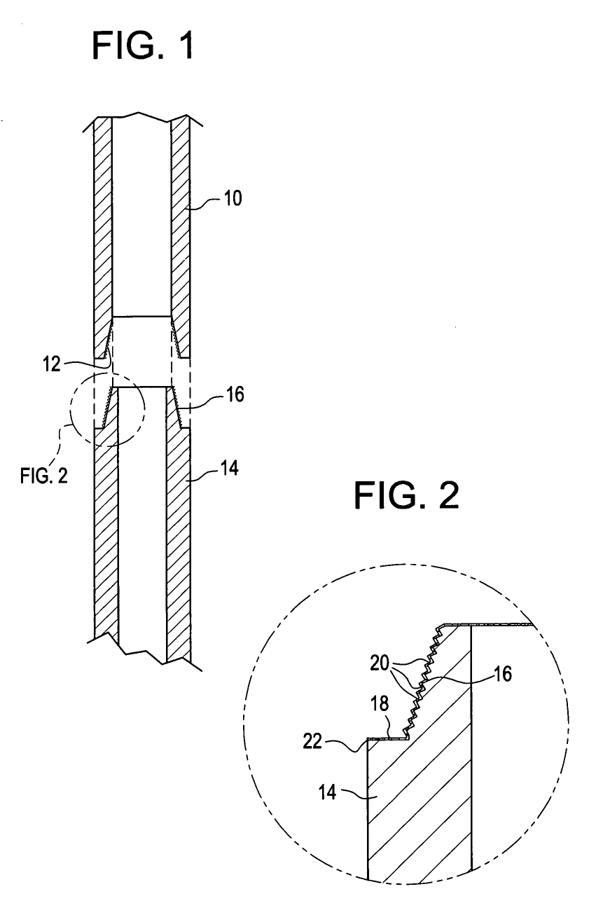

Referring to FIG. 1, one example of the application of the compositions disclosed herein is illustrated. A pipe 10 having a box thread 12, and a pipe 14 having a pin thread 16, are illustrated. It will be understood that this illustration is but one example of a set of mating parts that may be treated by the composition and method hereof. The coating composition is applied to pin thread 16. The coating could be applied to the box thread 12 instead if desired. In one embodiment, application of the composition is via spray. For ease of application, this may be on each exposed surface of pin thread 16, although it is to be noted that only surfaces subject to contact by a mating member and/or corrosion need be coated. The coating is illustrated schematically in FIG. 2. It will be appreciated that coating 18 is present on all surfaces that will contact pipe 10 when pipe 14 is mated therewith. This includes all thread flanks 20 and shoulder 22 as the purpose of coating 18, among other things discussed hereunder, includes preventing physical contact between the material of pipe 10 and pipe 14.

In this particular example, the coefficient of friction noted above is helpful in the makeup of pipe joints for the downhole industry. The high makeup torques based upon the desired coating friction factor of 0.08 help to provide for a high torque capacity to prevent over torquing of connections and breaking (i.e., unmaking) of connections during service in the well.

In the example shown, it is noted that some downhole devices, especially in the hydrocarbon exploration and recovery art, include electric communication relay conductors. Such conductors could easily short if contacted by a conductive compound. The coating composition and thread dope hereof therefore are in some embodiments non-conductive in nature and present no difficulty with respect to making contact with communication conductors or other conductors exposed to the coating or dope.

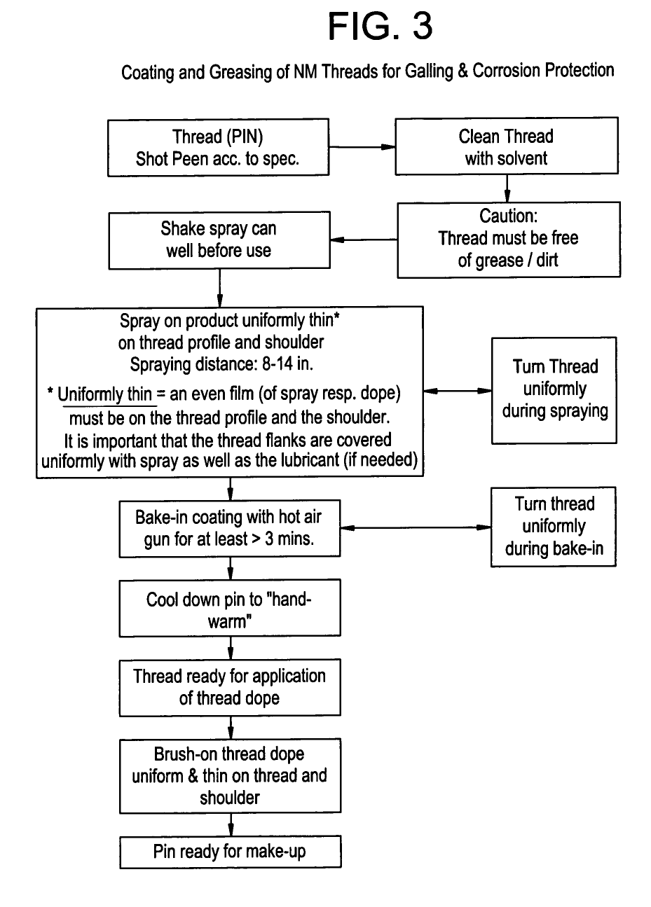

With the drawing FIG. 1 still in mind, one example of a specific process by which the coating composition may be applied is disclosed and illustrated in a flow diagram (see FIG. 3). Broadly stated, that process requires the cleaning of the contact areas to be coated; and applying the coating composition onto the target surface(s) in a uniform and thin manner. Uniformity is important as it ensures that all of the discrete areas of the contact surface are covered and there are no missed spots. The thinness is important to avoid distortion of the thread profile (as noted above) so that the contact areas engage each other smoothly as intended when they are made up. The process may also include hardening the coating.

Returning to the specific process illustrated in FIG. 3, the thread surface (or other contact area) is shot-peened to remove undesirable material. The thread (or other contact area) is then cleaned with a solvent capable of removing grease and dirt. With the surface prepared, the coating is sprayed onto the target surfaces in a uniform coating as thinly as can be applied while maintaining uniformity and cohesiveness of the coating. Once a uniform coating is applied the coating is subjected to hot air from a standard commercially available heat gun for about 3 minutes to accelerate solvent evaporation and to accelerate cross-linking to harden the coating. Appropriately the heat should be evenly distributed about the coated surface. If a dope is also to be applied, the heated surface is to be cooled until it is easily handled with respect to temperature of the surface. At this point dope may be applied by such as brushing. The dope is to be uniformly applied and thin while maintaining full coverage of the target surface. The thread is ready for engagement with a complementary thread.

While preferred embodiments have been shown and described, modifications and substitutions may be made thereto without departing from the spirit and scope of the invention. Accordingly, it is to be understood that the present invention has been described by way of illustrations and not limitation.

What is claimed:

1. A method for controlling galling comprising:
applying a coating comprising about 3.5 to about 36 wt %, based upon the total weight of the composition, of a fluoridic or oxidic compound of a transition of alkaline earth metal; about 4 to about 96.5 wt %, based upon the total weight of the composition of an adherent; about 0 to about 65 wt % to a contact area of a member.

2. A method for controlling galling as claimed in claim 1 wherein said compound is calcium fluoride.

3. A method for controlling galling as claimed in claim 1 wherein said adherent is a phenolic resin.

4. A method for controlling galling as claimed in claim 1 wherein said applying is spraying.

5. A method for controlling galling as claimed in claim 4 wherein said applying further includes heating the sprayed coating on the contact area.

6. A method for controlling galling as claimed in claim 1 wherein said applying is one of painting, dipping, electrostatic coating.

7. A method for controlling galling as claimed in claim 1 wherein said applying is by electrophoresis.

8. A method for controlling galling as claimed in claim 1 wherein said applying includes hardening said composition on said threaded member.

9. A method for controlling galling as claimed in claim 1 wherein said method includes applying a thread dope to the contact area over the coating.

10. A method for controlling galling as claimed in claim 9 wherein said thread dope includes an antigalling agent.

11. A method for controlling galling as claimed in claim 10 wherein said antigalling agent is calcium fluoride.

12. A method for controlling galling as claimed in claim 9 wherein said applying thread dope is by one of spraying, painting, dipping and electrostatic coating.

13. A method for controlling galling as claimed in claim 1 wherein said adherent is rendered effective by mixing with an effective amount of solvent.

14. A method for controlling galling as claimed in claim 1 wherein said method further includes applying thread dope to said contact area on said member.

15. A method for controlling galling as claimed in claim 1 wherein said connecting includes overcoming a coefficient of friction of about 0.08μ.

16. A gall resistant threaded member comprising:
a threaded member;
a coating disposed at threads of said threaded member, said coating containing about 90 wt % based on the total weight of the coating of a fluoridic or oxidic compound of a transition or alkaline earth metal and about 10 wt % of an adherent based on the total weight of the coating.

17. A gall resistant threaded member as claimed in claim 16 wherein said compound is calcium fluoride.

18. A gall resistant threaded member as claimed in claim 16 wherein said adherent is a phenolic resin.

19. A gall resistant threaded member as claimed in claim 16 wherein said member further includes a thread dope containing calcium fluoride.

20. A gall resistant threaded member as claimed in claim 16 wherein said coating is uniform and sufficiently thin to avoid distorting a profile of the thread.

21. A gall resistant threaded member as claimed in claim 16 wherein said coating material provides a coefficient of friction of about 0.08μ (mu).

22. A gall resistant threaded member as claimed in claim 16 wherein said coating is a sprayed coating.

23. A gall resistant threaded member as claimed in claim 16 wherein said coating is applied by electrophoresis.

24. An antigalling coating composition comprising:
about 3.5 to about 36 wt %, based upon the total weight of the composition, of a fluoridic or oxidic compound of a transition of alkaline earth metal;
about 4 to about 96.5 wt %, based upon the total weight of the composition of an adherent; and
about 0 to about 65 wt %, based upon the total weight of the composition of a solvent.

25. An antigalling coating as claimed in claim 24 wherein said compound is calcium fluoride.

26. An antigalling coating as claimed in claim 24 wherein said adherent is a phenolic resin.

27. An antigalling coating as claimed in claim 24 wherein said solvent is alcohol.

28. An antigalling coating as claimed in claim 24 wherein said composition is hardenable.

29. An antigalling coating as claimed in claim 28 wherein said composition hardens due to cross-linking and has a shear strength of about 10 Mega Pascals.

30. An antigalling coating as claimed in claim 24 wherein said composition is non-conductive.

31. An antigalling coating as claimed in claim 24 wherein said composition further includes a corrosion inhibitor.

32. An antigalling coating composition comprising:
an effective amount of a fluoridic or oxidic compound of a transition of alkaline earth metal to reduce galling;
an effective amount of an adherent to adhere the composition to a target surface; and
an effective amount of a solvent to facilitate application of the composition.

33. A method for applying an antigalling coating, the coating having an effective amount of a fluoridic or oxidic compound of a transition of alkaline earth metal to reduce galling, an effective amount of an adherent to adhere the composition to a target surface, an effective amount of a solvent to promote application of the composition, comprising:
cleaning a target area to be coated; and
applying the coating.

34. A method as claimed in claim 33 wherein said cleaning includes shot-peening.

35. A method as claimed in claim 33 wherein said cleaning includes application of a cleaning solvent.

36. A method as claimed in claim 33 wherein said applying is by electrophoresis.

37. A method as claimed in claim 33 wherein said applying includes spraying said coating onto the target area.

38. A method as claimed in claim 37 wherein the method further includes hardening the coating.

39. A method as claimed in claim 38 wherein the hardening includes baking of the coating.

40. A method as claimed in claim 39 wherein the baking is directing hot air toward the coating.

41. A method as claimed in claim 33 wherein the applying is carried out such that the coating is uniformly distributed over the target area.

42. A method as claimed in claim 41 wherein the uniform distribution is cohesive.

43. A method as claimed in claim 41 wherein the uniform distribution is sufficiently thin to avoid distorting the target area.

44. A method as claimed in claim 43 wherein the target area is a thread profile.
